(19) United States
(12) Patent Application Publication
(10) Pub. No.: US 2004/0167481 A1
(43) Pub. Date: Aug. 26, 2004

(54) Hand-held buffing device

(75) Inventors: Vito James Carlucci, Stratford, CT (US); Martin A. Cohen, Ridgefield, CT (US); Robert A. Staub JR., Ridgefield, CT (US); Albert J. George, Beacon Falls, CT (US)

Correspondence Address: Charles N.J. Ruggiero, Esq., Ohlandt, Greeley, Ruggiero & Perle, L.L.P., 10th Floor, One Landmark Square, Stamford, CT, 06901-2682, US

(73) Assignee: CONAIR CORPORATION

(21) Appl. No.: 10/758,243

(22) Filed: Jan. 15, 2004

Related U.S. Application Data
(60) Provisional application No. 60/440,100, filed on Jan. 16, 2003.

(51) Int. Cl. A61F 7/00

(52) U.S. Cl. 604/291

(57) ABSTRACT

A hand-held buffing device having a body with at least two portions, a handle portion and a head portion is provided. The handle portion is preferably ergonomically shaped and/or oriented for convenient, comfortable handling and may preferably have a control interface for allowing an operator to control the operation of buffing device. The head portion may preferably accommodate at least a portion of a drive assembly and operatively connect with one or more buffing pads or disks that are driven by the drive assembly to move in relatively rapid, reciprocating movements in short strokes in simultaneously different directions for hair removal, massaging and exfoliating with efficiency. The buffing device enables relatively lightweight motors and components to be used.

### BACKGROUND OF THE INVENTION

1. Field of the Invention

The present invention relates to handheld personal care appliances. More particularly, the present invention relates to an electric-powered handheld buffing device for removal of hair, smoothing of skin, and massaging of skin and muscles.

2. Description of the Related Art

Devices exist for hair removal and for skin smoothing. Typical hair removal devices grab and pull individual hairs or small groups of hairs. These devices typically require complex construction of small, moving parts and they take a relatively long time to operate in terms of hair removal. Certain of such devices are painful to use and may result in infected follicles. Various such devices have been described in U.S. Pat. Nos. 5,281,233, 5,057,115 and 4,935,024. Typical skin smoothing devices typically utilize a sponge-like, loofah, or other roughened surface that is suitable for being manually or automatically moved relative to and against the skin of a person. Such skin smoothing devices are not intended to remove hair and are not well suited for such an operation. Accordingly, there is a recognized need for an efficient, inexpensive hair remover that gently removes unwanted hair without irritating the skin, and exfoliates skin leaving skin smooth, silky, and healthy.

### SUMMARY OF THE INVENTION

It is an object of the present invention to provide an improved hand-held buffing device for comfortably and efficiently removing body hair and exfoliating skin.

It is another object of the present invention to provide an improved hand-held buffing device that provides for buffing movements that are efficient in terms of load to a drive assembly.

It is yet another object of the present invention to provide an improved hand-held buffing device that has replaceable and/or interchangeable buffing pads.

It is still another object of the present invention to provide an improved hand-held buffing device that is ergonomically shaped for easy handling and/or operation.

It is a further object of the present invention to provide an improved hand-held buffing device that has and/or operatively cooperates with a variety of ergonomically shaped, sized, and/or configured buffing pads for effectively and efficiently interacting with various parts of a body.

These and other objects and advantages of the present invention are achieved by an improved hand-held buffing device having a handle portion preferably with a control interface, a head portion preferably with a drive assembly, and one or more buffing pads that may be operatively connected to the drive assembly. The buffing pads may have any of a variety of shapes, sizes and/or configurations.

### BRIEF DESCRIPTION OF THE DRAWINGS

Other and further objects, advantages and features of the present invention will be understood by reference to the following specification in conjunction with the accompanying drawings, in which like reference characters denote like elements of structure.

FIG. 1 is a schematic first side view of a hand-held buffing device in accordance with an illustrative embodiment of the present invention;

FIG. 2 is a schematic second side view, rotated about 90 degrees from the first side view of the hand-held buffing device of FIG. 1;

FIG. 3 is a schematic plan view of a buffing pad in accordance with an illustrative embodiment of the present invention;

FIG. 4 is a schematic plan view of a buffing pad in accordance with another illustrative embodiment of the present invention;

FIG. 5 is a schematic plan view of a buffing pad in accordance with yet another illustrative embodiment of the present invention;

FIG. 6 is a schematic plan view of a buffing pad in accordance with still another illustrative embodiment of the present invention;

FIG. 7 is a schematic plan view of a buffing pad in accordance with a further illustrative embodiment of the present invention;

FIG. 8 is a schematic plan view of a buffing pad in accordance with still a further illustrative embodiment of the present invention;

FIG. 9 is a schematic side view of a hand-held buffing device in accordance with another illustrative embodiment of the present invention;

FIG. 10 is a schematic side view of a hand-held buffing device in accordance with still another illustrative embodiment of the present invention;

FIG. 11 is a schematic side view of a hand-held buffing device in accordance with yet another illustrative embodiment of the present invention;

FIG. 12 is a schematic side view of a hand-held buffing device in accordance with yet still another illustrative embodiment of the present invention;

FIG. 13 is a schematic perspective view of rollers that may be associated with the hand-held buffing device of FIG. 13;

FIG. 14 is a schematic perspective view of belts that may be associated with the hand-held buffing device of FIG. 13;

FIG. 15 is a schematic side view of a hand-held buffing device in accordance with a further illustrative embodiment of the present invention;

FIG. 16 is a schematic side view of a hand-held buffing device in accordance with another embodiment of the present invention;

FIG. 17 is a schematic side view of a hand-held buffing device in accordance with still another embodiment of the present invention;

FIG. 18 is a schematic side view of a hand-held buffing device in accordance with yet another embodiment of the present invention;

FIG. 19 is a schematic side view of a hand-held buffing device in accordance with yet still another embodiment of the present invention;

FIG. 20 is a schematic end view of a head portion that may be associated with any of the various embodiments of the present invention; and

FIG. 21 is a schematic perspective view of an alternative head portion that may be associated with any of the various embodiments of the present invention.

### DETAILED DESCRIPTION OF THE INVENTION

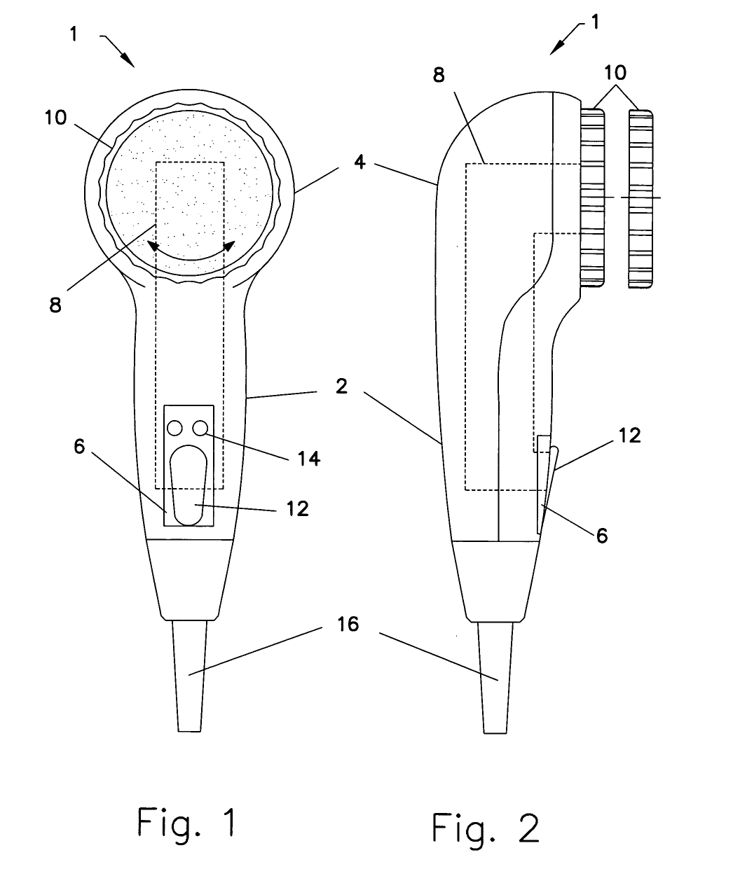

Referring to the drawings and in particular to FIGS. 1 and 2, there is shown a hand-held buffing device in accordance with a preferred embodiment of the present invention generally represented by reference numeral 1. Buffing device 1 has at least two portions, a handle portion 2 and a head portion 4. The handle portion 2 is preferably ergonomically shaped and/or oriented for convenient, comfortable handling. The handle portion 2 may preferably have a control interface 6 for allowing an operator to control the operation of buffing device 1. The head portion 4 may preferably accommodate at least a portion of a drive assembly 8 and operatively connect with one or more buffing pads 10.

As reflected in FIG. 2, control interface 6 may preferably be operatively connected to drive assembly 8, and, the drive assembly may preferably be operatively connected to buffing pads 10. Control interface 6 may preferably have any of a variety of shapes, sizes and/or configurations. For example, as shown in FIG. 1, control interface 6 may have any of a variety of input selectors 12 (e.g., buttons, knobs, dials, touch-pads or touch-screens, etc.) for starting, stopping and/or otherwise adjusting the operation of buffing device 1. Control interface 6, in one or more different aspects of the present invention, may also be provided with any of a variety of output indicators 14, such as, for example, visual indicators (e.g., LED/LCD displays, etc.), audible indicators (e.g., beeps, automated voice, etc.), or tactile indicators (e.g., vibration, heat, etc.). Thus, control interface 6 may preferably cooperate with input selectors 12 and/or output indicators 14 to facilitate operative communication between the operator and buffing device 1.

Drive assembly 8 is generally shown in phantom lines to demonstrate that it is housed by handle portion 2 and/or head portion 4 and to reflect that it may have any of a variety of configurations and/or components. For example, drive assembly 8 may have a drive shaft (not shown) operatively connected to a drive motor (not shown) for generating linear and/or rotational motion. In turn, the drive shaft may be either directly or indirectly operatively connected to one or more buffing pads 10 to provide the appropriate type and/or degree of motion thereto for comfortably massaging and efficiently removing body hair and exfoliating skin. Other configurations and/or components may also be used to provide the necessary motion to one or more buffing pads 10. As will be discussed in more detail later, the type and/or amount of motion required may depend on the configuration of the particular buffing pad 10 employed. It is noted that buffing device 1 is preferably electrically powered. It may be battery operated or run from an electric cord 16 connected to an electric power source.

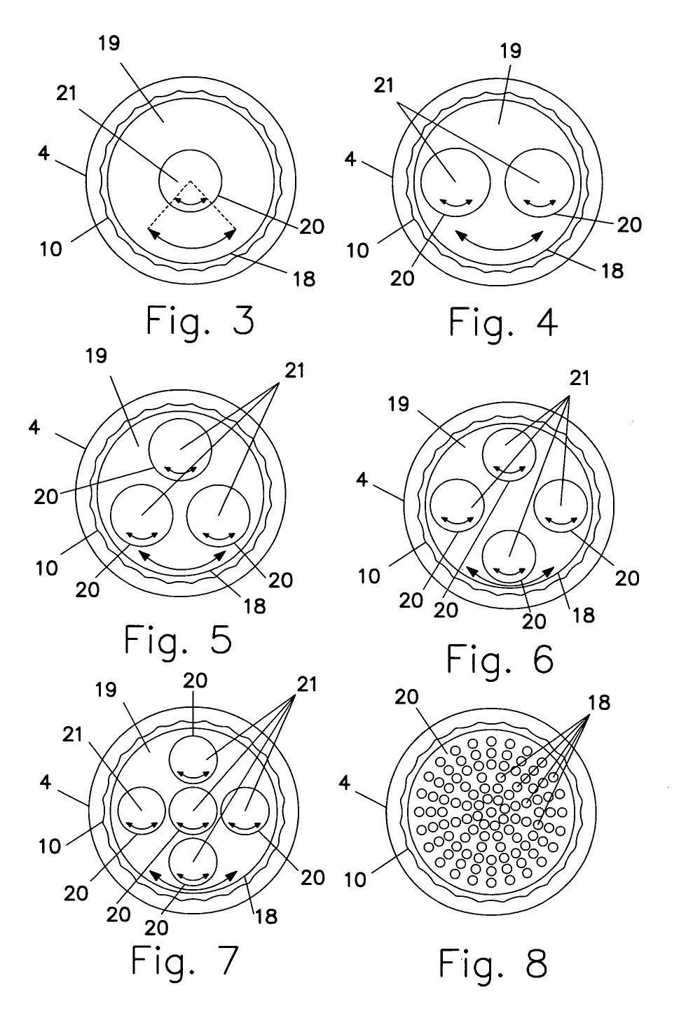

Referring to FIGS. 3 through 8, in certain embodiments of the present invention, one or more buffing pads 10 may have two or more disk pads, such as, for example, at least one outer disk pad 18 and at least one inner disk pad 20. Preferably, drive assembly 8 is operatively connected to both outer disk pad 18 and inner disk pad 20 so that each may be independently operated. For example, outer disk pad 18 may be rotatably operated in a first direction while inner disk pad 20 is rotatably operated in an opposite, second direction, simultaneously.

In one aspect of the present invention, neither disk pad 18, 20 moves or operates in a complete revolution, but instead each moves in a predefined arc of preferably between about 30 degrees to about 70 degrees. Preferably, upon moving through the predefined arc, disk pads 18, 20 reverse direction and move in the same arc in the opposite direction. Thus, each disk pad 18, 20 may preferably move or operate in a repeating reciprocating manner. In another aspect of this embodiment, either or both disk pad 18, 20 may selectively move or operate in complete revolutions.

Thus, in operation, buffing device 1 may preferably be operated by holding one or more buffing pads 10 in contact with the skin so that when disk pads 18, 20 move or are operated in different directions, preferably simultaneously, either in short reciprocating strokes or complete revolutions, body hair may be gently twisted and broken off. As each disk pad 18, 20 is preferably provided with an abrasive surface 19, 21 that may be, for example, roughened rubber, sand-paper, sponge or loofah, or any other known abrasive suitable for treating skin, abrasive surface 19, 21 preferably facilitates gently engaging, twisting, and breaking off body hair. For most efficient exfoliating and hair removal, it is desirable to use sand-paper type surfaces. Each abrasive surface 19, 21 may also be suitable for massaging muscles and removing calluses.

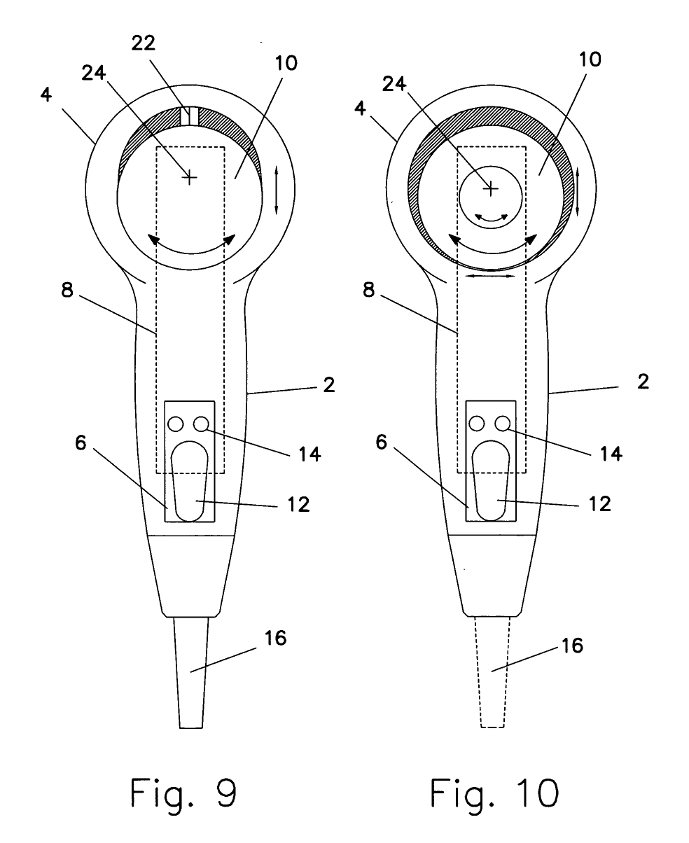

Referring to FIGS. 9 through 11, in other embodiments of the present invention, buffing pads 10, in addition to having multiple, moving disk pads 18, 20 that may preferably operate in different rotational directions simultaneously, may preferably cooperate with one or more tracks 22 to facilitate one or more additional degree of motion. Tracks 22 may be part of head portion 4 and/or drive assembly 8, or may be part of buffing pads 10. As shown in FIG. 9, track 22 may be linear so that disk pads 18, 20 may move linearly there along, preferably in a reciprocating manner, while rotating in arcuate paths relative to one another.

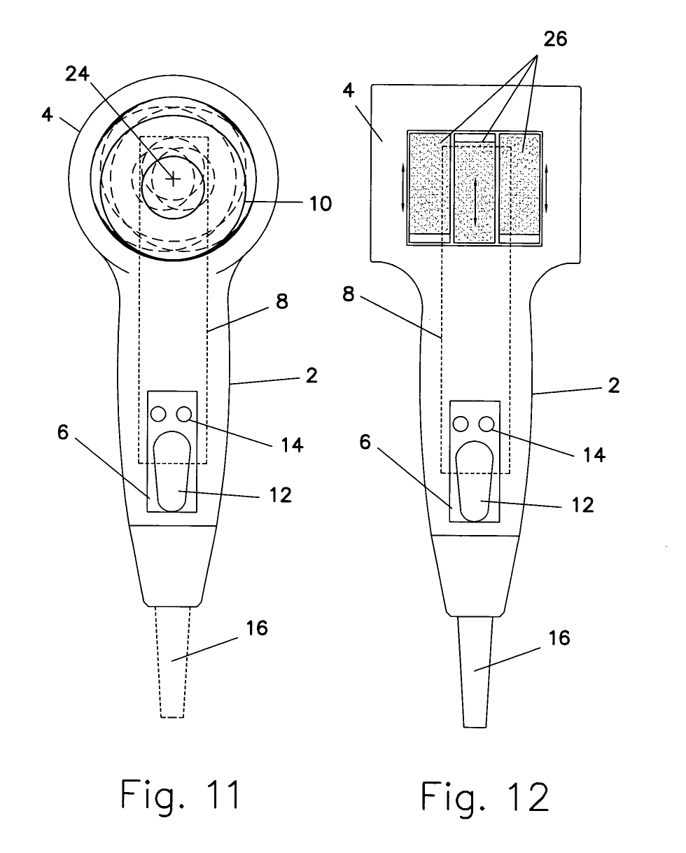

In other aspects of the present invention, tracks 22 may have different configurations enabling a variety of different motions. For example, as shown in FIGS. 10 and 11, disk pads 18, 20 may move in multiple directions, preferably in a reciprocating manner, with respect to a center point 24 of head portion 4. Thus, in certain embodiments of the present invention, buffing pads 10 and/or disk pads 18, 20 may be rotatable in multiple directions while simultaneously reciprocating alternatively between multiple linear directions or eccentrically about center point 24.

Referring to FIG. 12, in another embodiment of the present invention, buffing pads 10 may be in the form of two or more square or rectangular pads 26. Preferably, drive assembly 8 is operatively connected to each rectangular pad 26 so that each may be independently operated. For example, as shown, each rectangular pad 26 may be linearly operated in a reciprocating manner.

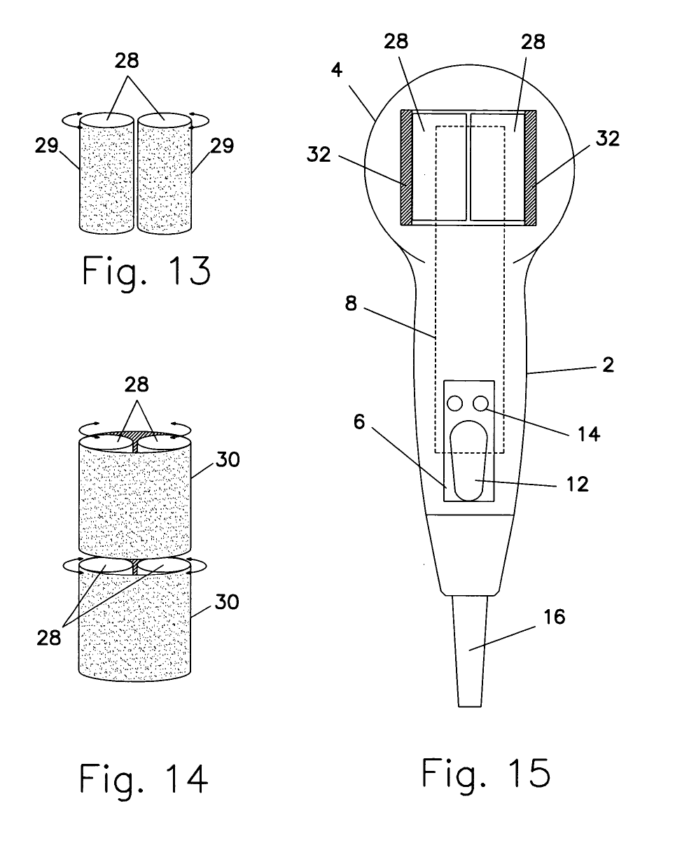

Referring to FIGS. 13 through 14, buffing pads 10 may be in the form of one or more rotating rollers 28. As shown in FIG. 13, rotating rollers 28 may each have an abrasive surface 29. Alternatively, as shown in FIG. 14, rotating rollers 28 may support and/or cause to rotate one or more abrasive belts 30 that move along with rotating rollers 28 accordingly. In one aspect of the invention, two or more belts 30 may be placed side-by-side and rotated in opposite directions. Preferably, drive assembly 8 is operatively connected to each rotating roller 28 so that the rotating rollers may reversibly operate or rotate in complete revolutions and/or in arcuate, reciprocating paths.

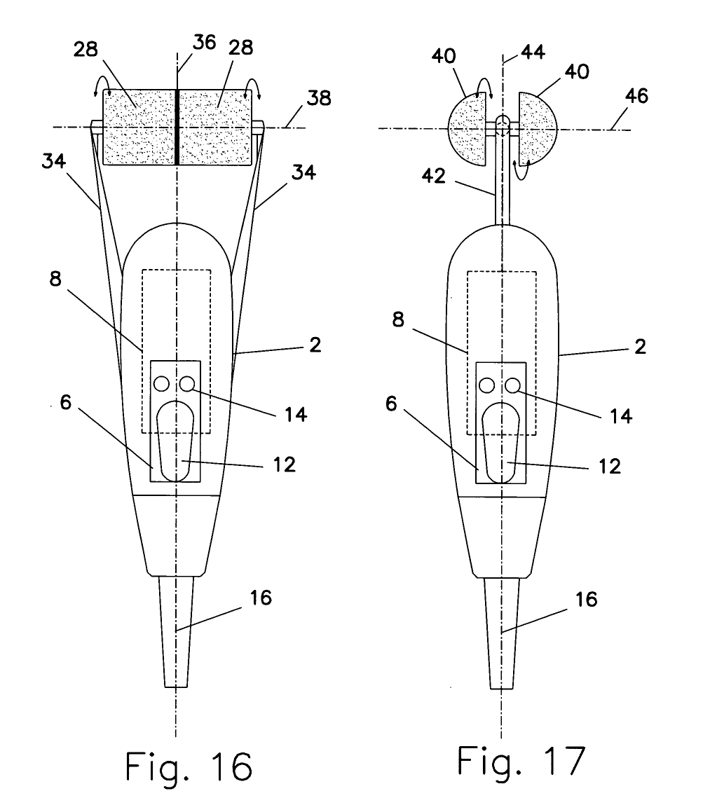

As shown in FIG. 15, buffing device 1 may also be provided with one or more wipers 32 for removing hair, skin and moisture from rollers 28 and/or belts 30 during operation. As shown in FIG. 16, in still another aspect of the present invention, buffing device 1 may have a pair of supports or arms 34 for rotationally supporting one or more rollers 28 for rotation about an axis 36 preferably at least somewhat perpendicular to a longitudinal axis 38 of the handle portion 2. Each roller 28 may preferably rotate in complete revolutions or, alternatively, in a reciprocating manner changing directions in an arcuate path.

Referring to FIGS. 17 through 19, in still other embodiments of the present invention, head portion 4 may itself take the form of one or more abrasive buffing heads 40. For example, as shown in FIG. 17, buffing device 1 may have one or more abrasive buffing heads 40 that are semi-spherical and rotationally-mounted via at least one mounting structure 42. In this aspect of the present invention, abrasive buffing heads 40 preferably rotate about an axis 44 preferably at least somewhat perpendicular to a longitudinal axis 46 of the handle portion 2. Each abrasive buffing head 40 may preferably rotate in complete revolutions or, alternatively, in a reciprocating manner changing directions in an arcuate path.

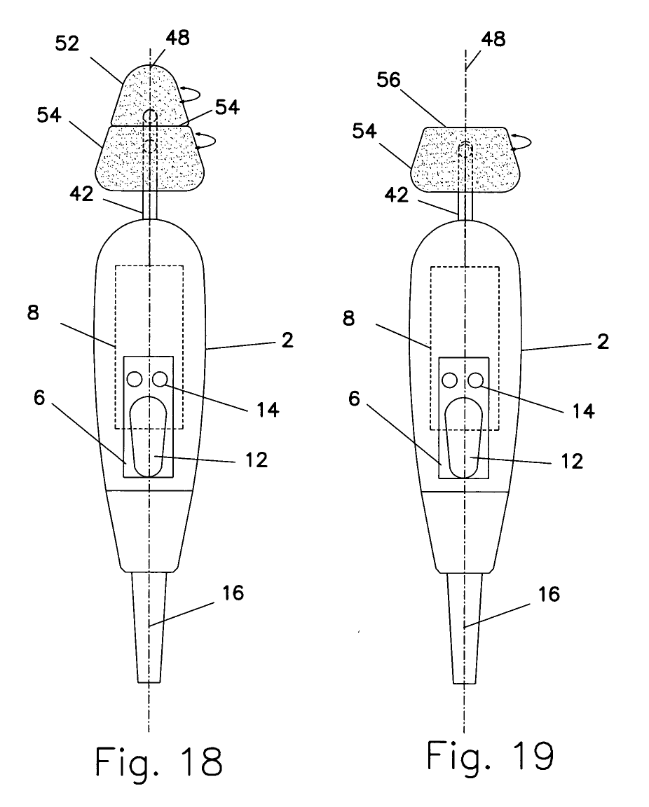

Alternatively, as shown in FIGS. 18 and 19, buffing device 1 may have one or more abrasive buffing heads 40 that are conical and rotationally-mounted via at least one mounting structure 42. In this aspect of the present invention, abrasive buffing heads preferably rotate about an axis 48 preferably at least somewhat parallel to a longitudinal axis 50 of the handle portion 2. Each abrasive buffing head may preferably rotate in complete revolutions or, alternatively, in a reciprocating manner changing directions in an arcuate path. It is noted that abrasive buffing heads 52, 54 may have a variety of shapes and/or sizes. For instance, a first abrasive buffing head 52 may have a rounded point for enabling hair removal or massage in tight corners of the anatomy while a second abrasive buffing head 54 may have a flat, nose portion 56 and a cone-wall profile that is continuous with the first abrasive buffing head 52 to form a smooth cone side-wall. Then again, either first or second abrasive buffing heads 52, 54 may stand alone as shown in FIG. 19 with respect to the second abrasive buffing head 54.

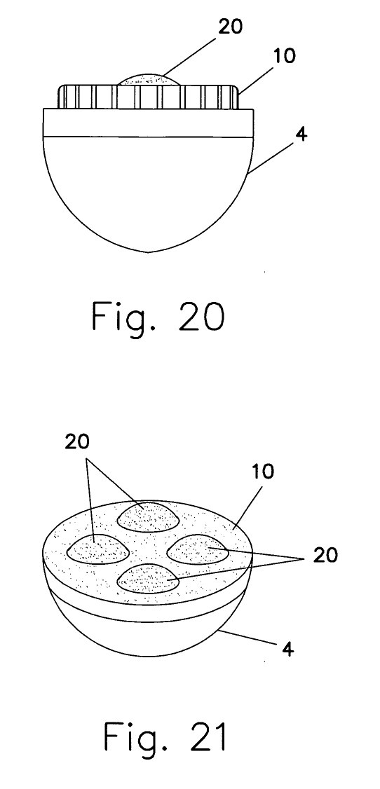

The present invention having been thus described with particular reference to the preferred forms thereof, it will be obvious that various changes and modifications may be made therein without departing from the spirit and scope of the present invention as defined herein. For example, as shown in FIGS. 20 and 21, buffing pads 10 may have one or more raised inner disk pads 20 for enhancing the ability to buff irregular body contours. It is believed that the many advantages of this invention and the manner in which it fulfills the stated objectives will be understood by those skilled in the art.

What is claimed is:

1. A buffing device comprising:
a handle portion with at least a portion of a control interface;
a head portion with at least a portion of a drive assembly operatively connected to said control interface; and
one or more buffing pads being operatively connected to said drive assembly,
wherein said one or more buffing pads releasably connect to said drive assembly.

2. The buffing device of claim 1, wherein said one or more buffing pads have an abrasive surface.

3. The buffing device of claim 1, wherein said drive assembly causes said one or more buffing pads to move in one or more directions.

4. The buffing device of claim 1, wherein said drive assembly causes said one or more buffing pads to rotate in one or more directions.

5. The buffing device of claim 1, wherein said drive assembly causes said one or more buffing pads to move in an arcuate, reciprocating manner.

6. The buffing device of claim 1, wherein said drive assembly causes said one or more buffing pads to move in at least one rotational direction and at least one linear direction.

7. The buffing device of claim 1, wherein said one or more buffing pads have one or more raised portions.

8. The buffing device of claim 7, wherein said one or more raised portions move independently in one or more directions.

9. The buffing device of claim 7, wherein said one or more buffing pads and said one or more raised portions move independently in one or more directions.

10. A skin buffing device comprising:
a handle portion with at least a portion of a control interface;
a head portion with at least a portion of a drive assembly operatively connected to said control interface; and
one or more buffers being operatively connected to said drive assembly,
wherein said one or more rollers releasably connect to said drive assembly.

11. The skin buffing device of claim 10, wherein said one or more rollers have an abrasive surface.

12. The skin buffing device of claim 10, wherein said drive assembly causes said one or more rollers to rotate in one or more directions.

13. The skin buffing device of claim 10, wherein said one or more rollers support and cause to rotate one or more abrasive buffing belts.

14. The skin buffing device of claim 10, further comprising one or more wipers for removing debris from said one or more rollers.

15. The buffing device of claim 14, wherein said one or more wipers are flexible strips proximately positioned adjacent said one or more rollers.

16. A buffing device comprising:
a handle portion accommodating at least one drive assembly and at least one operating control;
one or more buffing heads; and
one or more arms operatively connecting said one or more buffing heads to said at least one drive assembly.

17. The buffing device of claim 16, wherein said one or more buffing heads are selected from a group consisting of one or more abrasive rollers, one or more abrasive semi-spherical members, or one or more abrasive conical members, or any combination of the same.

18. The buffing device of claim 16, wherein said drive assembly causes said one or more buffing heads to move in one or more directions.

19. The buffing device of claim 16, wherein said drive assembly causes said one or more buffing heads to rotate in one or more directions.

20. The buffing device of claim 16, wherein said one or more buffing heads rotate about a first axis at least somewhat perpendicular to a longitudinal axis of said handle portion and/or about a second axis at least somewhat parallel to said longitudinal axis of said handle portion.
